(19) United States
(12) Patent Application Publication
(10) Pub. No.: US 2006/0105016 A1
(43) Pub. Date: May 18, 2006

(54) Device compatible with magnetic resonance imaging

(76) Inventors: Robert W. Gray, Rochester, NY (US); David Cope, Medville, MA (US); David A. Glocker, West Henrietta, NY (US)

Correspondence Address: HOWARD J. GREENWALD P.C., 349 W. COMMERCIAL STREET SUITE 2490, EAST ROCHESTER, NY, 14445-2408, US

(21) Appl. No.: 11/132,469

(22) Filed: May 19, 2005

Related U.S. Application Data
(60) Provisional application No. 60/627,716, filed on Nov. 12, 2004.

(51) Int. Cl. A61N 1/00; A61F 2/00

(52) U.S. Cl. 424/423; 607/2; 977/931

(57) ABSTRACT

A plurality of coated layers is disposed on an implanted device. The materials and electrical parameters of the coated layers are chosen and the geometry of the coated layers is arranged so that incident electromagnetic radiation induces currents in the coated layers that have a predetermined phase and amplitude relationship with the current induced in the implanted device.

### CROSS-REFERENCE TO RELATED APPLICATIONS

This application claims the benefit of U.S. Provisional Application No. 60/627,716 filed Nov. 12, 2004.

### FIELD OF THE INVENTION

This invention relates to the field of Magnetic Resonance Imaging and more particularly to imaging of implanted devices and the biological tissue in the vicinity of such implanted devices.

### BACKGROUND OF THE INVENTION

Magnetic Resonance Imaging (MRI) is extensively used to non-invasively diagnose patient medical problems. The patient is positioned in the aperture of a large annular magnet that produces a strong and static magnetic field. The spins of the atomic nuclei of the patient's tissue molecules are aligned by the strong static magnetic field. Radio frequency pulses are then applied in a plane perpendicular to the static magnetic field lines so as to cause some of the hydrogen nuclei to change alignment. The frequency of the radio wave pulses used is governed by the Larmor Equation. Magnetic field gradients are then applied in the 3 dimensional planes to allow encoding of the position of the atoms. At the end of the radio frequency pulse the nuclei return to their original configuration and, as they do so, they release radio frequency energy, which can be picked up by coils wrapped around the patient. These signals are recorded and the resulting data are processed by a computer to generate an image of the tissue. Thus, the examined tissue can be seen with its quite detailed anatomical features. In clinical practice, MRI is used to distinguish pathologic tissue such as a brain tumor from normal tissue.

The technique most frequently relies on the relaxation properties of magnetically-excited hydrogen nuclei in water. The sample is briefly exposed to a burst of radiofrequency energy, which in the presence of a magnetic field puts the nuclei in an elevated energy state. As the molecules undergo their normal, microscopic tumbling, they shed this energy to their surroundings, in a process referred to as “relaxation.” Molecules free to tumble more rapidly relax more rapidly.

T1-weighted MRI scans rely on relaxation in the longitudinal plane, and T2 weighted MRI scans rely on relaxation in the transverse plane. Differences in relaxation rates are the basis of MRI images—for example, the water molecules in blood are free to tumble more rapidly, and hence, relax at a different rate than water molecules in other tissues. Different scan sequences allow different tissue types and pathologies to be highlighted.

MRI allows manipulation of spins in many different ways, each yielding a specific type of image contrast and information. With the same machine a variety of scans can be made and a typical MRI examination consists of several such scans.

One of the advantages of a MRI scan is that, according to current medical knowledge, it is harmless to the patient. It only utilizes strong magnetic fields and non-ionizing radiation in the radio frequency range. Compare this to CT scans and traditional X-rays which involve doses of ionizing radiation. It must be noted, however, that the presence of a ferromagnetic foreign body (say, shell fragments) in the patient, or a metallic implant (like surgical prostheses, or pacemakers) can present a (relative or absolute) contraindication towards MRI scanning: interaction of the magnetic and radiofrequency fields with such an object can lead to mechanical or thermal injury, or failure of an implanted device.

Even if implanted medical devices pose no danger to the patient, they may prevent a useful MR image from being obtained, due to their perturbation of the static, gradient and/or radio frequency pulsed magnetic fields and/or the response signal from the imaged tissue. Examples of problems encountered when attempting to use MRI to image tissue adjacent to implanted medical devices are discussed in U.S. Pat. No. 6,712,844, the entire disclosure of which is hereby incorporated by reference into this specification. U.S. Pat. No. 6,712,844 states, “While researching heart problems, it was found that all the currently used metal stents distorted the magnetic resonance images. As a result, it was impossible to study the blood flow in the stents which were placed inside blood vessels and the area directly around the stents for determining tissue response to different stents in the heart region.” U.S. Pat. No. 6,712,844 goes on to state “It was found that metal of the stents distorted the magnetic resonance images of blood vessels. The quality of the medical diagnosis depends on the quality of the MRI images. A proper shift of the spins of protons in different tissues produces high quality of MRI images. The spin of the protons is influenced by radio frequency (RF) pulses, which are blocked by eddy currents circulating at the surface of the wall of the stent. The RF pulses are not capable of penetrating the conventional metal stents. Similarly, if the eddy currents reduce the amplitudes of the radio frequency pulses, the RF pulses will lose their ability to influence the spins of the protons. The signal-to-noise ratio becomes too low to produce any quality images inside the stent. The high level of noise to signal is proportional to the eddy current magnitude, which depends on the amount and conductivity of the stent in which the eddy currents are induced and the magnitude of the pulsed field.”

The currents induced in implanted metallic stents, and other devices, by the incident radio frequency radiation in the MRI field create, according to Lenz's law, magnetic fields that oppose the change of the magnetic fields of the incident radiation, thereby distorting and/or reducing the contrast of the resulting image.

Examples of attempts to improve the image ability of stents in MRI by incorporating resonance circuits with the stents are found, i.e., in U.S. Pat. No. 6,280,385 (“Stent and MR Imaging Process for the Imaging and the Determination of the Position of a Stent”) and U.S. Pat. No. 6,767,360 (“Vascular Stent with Composite Structure for Magnetic Resonance Imaging Capabilities”). The entire disclosure of each of these United States patents is hereby incorporated by reference into this specification.

U.S. Pat. No. 6,280,385 states in column 3, lines 29-44: “These and other objects are achieved by the present invention, which comprises a stent which is to be introduced into the examination object. The stent is provided with an integrated resonance circuit which induces a changed response signal in a locally defined area in or around the stent that is imaged by spatial resolution. The resonance frequency is essentially equal to the resonance frequency of the applied high-frequency radiation of the magnetic resonance imaging system. Since that area is immediately adjacent to the stent (either inside or outside thereof), the position of the stent is clearly recognizable in the correspondingly enhanced area in the magnetic resonance image. Because a changed signal response of the examined object is induced by itself, only those artifacts can appear that are produced by the material of the stent itself.” Claim 1 in column 12 of U.S. Pat. No. 6,280,385 claims: “1. A magnetic resonance imaging process for the imaging and determination of the position of a stent introduced into an examination object, the process comprising the steps of: placing the examination object in a magnetic field, the examination object having a stent with at least one passive resonance circuit disposed therein; applying high-frequency radiation of a specific resonance frequency to the examination object such that transitions between spin energy levels of atomic nuclei of the examination object are excited; and detecting magnetic resonance signals thus produced as signal responses by a receiving coil and imaging the detected signal responses; wherein, in a locally defined area proximate the stent, a changed signal response is produced by the at least one passive resonance circuit of the stent, the passive resonance circuit comprising an inductor and a capacitor forming a closed-loop coil arrangement such that the resonance frequency of the passive resonance circuit is essentially equal to the resonance frequency of the applied high-frequency radiation and such that the area is imaged using the changed signal response.”

U.S. Pat. No. 6,767,360 states in column 2, lines 29-39: “Imaging procedures using MRI without need for contrast dye are emerging in the practice. But a current considerable factor weighing against the use of magnetic resonance imaging techniques to visualize implanted stents composed of ferromagnetic or electrically conductive materials is the inhibiting effect of such materials. These materials cause sufficient distortion of the magnetic resonance field to preclude imaging the interior of the stent. This effect is attributable to their Faraday physical properties in relation to the electromagnetic energy applied during the MRI process.” U.S. Pat. No. 6,767,360 further states in column 2, lines 50-64: “In German application 197 46 735.0, which was filed as international patent application PCT/DE98/03045, published Apr. 22, 1999 as WO 99/19738, Melzer et al (Melzer, or the 99/19738 publication) disclose an MRI process for representing and determining the position of a stent, in which the stent has at least one passive oscillating circuit with an inductor and a capacitor. According to Melzer, the resonance frequency of this circuit substantially corresponds to the resonance frequency of the injected high-frequency radiation from the magnetic resonance system, so that in a locally limited area situated inside or around the stent, a modified signal answer is generated which is represented with spatial resolution. However, the Melzer solution lacks a suitable integration of an LC circuit within the stent.”

Claims 1 and 2 in column 9 of U.S. Pat. No. 6,767,360 claim: “1. A stent adapted to be implanted in a duct of a human body to maintain an open lumen at the implant site, and to allow viewing body properties outside and within the implanted stent by magnetic resonance imaging (MRI) energy applied external to the body, said stent comprising a metal scaffold, and an electrical circuit resonant at the resonance frequency of said MRI energy integral with said scaffold. 2. A stent adapted to be implanted in a duct of a human body to maintain an open lumen at the implant site, said stent comprising a tubular scaffold of low ferromagnetic metal, and an inductance-capacitance (LC) circuit integral with said scaffold, said LC circuit being geometrically structured in combination with said scaffold to be resonant at the resonance frequency of magnetic resonance imaging (MRI) energy to be applied to said body to enable MRI viewing of body tissue and fluid within the lumen of the stent when implanted and subjected to said MRI energy.”

Both U.S. Pat. Nos. 6,280,385 and 6,767,360 teach the incorporation of LC resonant circuits with stents to improve the image ability of such stents in MRI. However, in addition to a resonant frequency, resonant circuits are characterized by a Q factor which is a measure of the bandwidth of the current peak amplitude at the resonant frequency and depends upon the total resistance R of the resonant circuit. If the Q factor is too high, indicating a highly tuned, narrow bandwidth and high peak current at resonance, the induced current in the circuit and resultant enhanced electromagnetic signal will cause the MR image to be too bright with accompanying loss of detail.

Applicants have discovered that image ability of stents may be optimized by incorporating RLC circuits with an optimized Q factor. In addition to the inductance L and capacitance C, resistance R must be selected for maximum image ability.

In light of the above, it is the object of the present invention to provide implantable devices that may be visualized by magnetic resonance imaging and further; to improve such imaging of tissue in the vicinity of such implanted devices.

### SUMMARY OF THE INVENTION

A plurality of coated layers is disposed on an implanted device. The material and electrical parameters of the coated layers are chosen and the geometry of the coated layers is arranged so that incident electromagnetic radiation induces currents in the coated layers that have a predetermined phase and amplitude relationship with the current induced in the implanted device.

### BRIEF DESCRIPTION OF THE DRAWINGS

In the detailed description of the preferred embodiments of the invention presented below, reference is made to the accompanying drawings, in some of which the relative relationships of the various components are illustrated, it being understood that orientation of the apparatus may be modified. For clarity of understanding of the drawings, relative proportions depicted or indicated of the various elements of which disclosed members are comprised may not be representative of the actual proportions, and some of the dimensions may be selectively exaggerated.

FIGS. 1A-C are schematic diagrams of stents according to the embodiments of the invention;

FIG. 2 is a cross-sectional view of a coated conducting ring assembly representing one embodiment of the invention;

FIG. 3 is a circuit diagram for an equivalent circuit model of the coated conducting ring assembly in FIG. 2;

FIG. 4 is a graph of model simulation results of currents induced in the coated conducting ring assembly of FIG. 2;

FIG. 5 is a cross-sectional view of a coated conducting ring assembly representing another embodiment of the invention;

FIG. 5A is a cross-sectional view of a coated conducting ring assembly similar to that of FIG. 5, but with the coatings encircling the ring twice.

FIG. 6 is a cross-sectional view of a coated conducting ring assembly representing yet another embodiment of the invention;

FIG. 7 is a cross-sectional view of a coated conducting ring assembly similar to the one in FIG. 2, but with the coatings on only the outer surface of the ring;

FIG. 8 is a cross-sectional view of a coated conducting ring assembly similar to the one in FIG. 5, but with the coatings on only the outer surface of the ring;

FIG. 8A is a cross-sectional view of a coated conducting ring assembly similar to the one in FIG. 5A, but with the coatings on only the outer surface of the ring;

FIG. 9 is a cross-sectional view of a coated conducting ring assembly similar to the one in FIG. 6, but with the coatings on only the outer surface of the ring;

FIG. 10 shows views, in perspective, of five different coating pattern embodiments;

FIGS. 11A and 11B are schematic diagrams of a portion of a device with a conducting surface having therein a hole with coatings according to embodiments of the invention surrounding the hole;

FIG. 12 is a schematic of coating layer embodiment on the framework of a stent;

FIG. 13 is a schematic of a method for determining the resonant frequency of a stent assembly; and

FIG. 14 is a block diagram of an apparatus for determining the resonant frequency of a stent assembly.

### DETAILED DESCRIPTION OF THE INVENTION

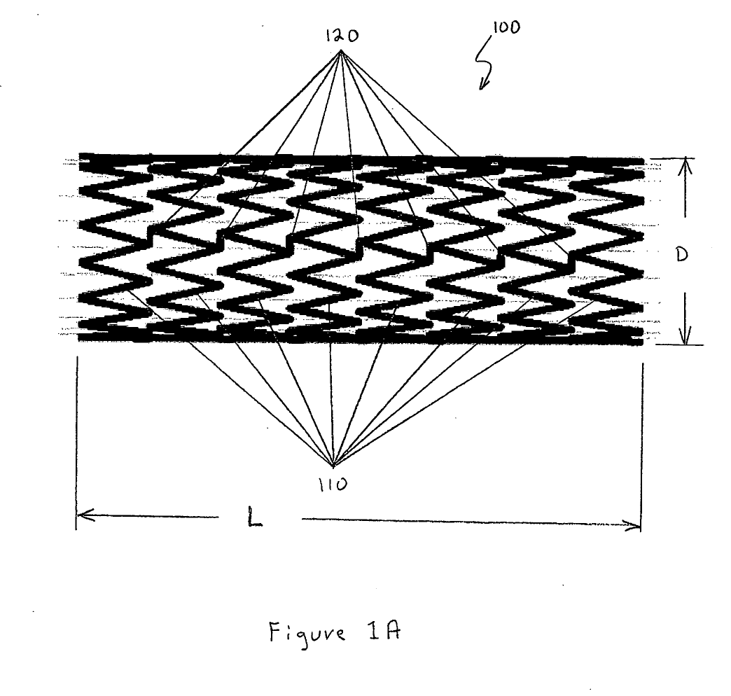

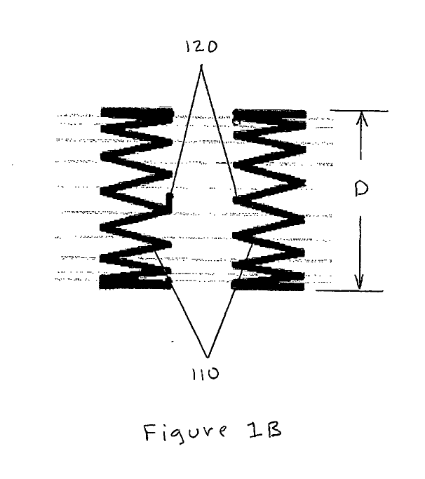

Referring to FIG. 1A, one embodiment is a coated stent. A stent is an expandable tubular mesh structure that is inserted into a lumen structure of the body to keep it open. Stents are used in diverse structures in the body such as the esophagus, trachea, blood vessels, and the like. Prior to use, a stent is collapsed to a small diameter. When brought into place it is expanded either by using an inflatable balloon or is self-expending due to the elasticity of the material. Once expanded the stent is held in place by its own tension. Stents are usually inserted by endoscopy or other procedures less invasive than a surgical operation. Stents are typically metallic, for example, stainless steel, alloys of nickel and titanium, or the like and are therefore electrically conducting.

FIG. 1A is a schematic illustration of one embodiment of a stent. FIG. 1A is a side elevational view of a tubular stent 100 having a length L and a diameter D. Stent 100 is comprised of a plurality of electrically conducting, sawtooth shaped circumferential loops 110, each loop 110 connected to the next loop 110 at a plurality of points 120 around the circumference of each loop 110. In stent 100 of FIG. 1A each loop 110 is connected to the next loop 110 at four points 120 around the circumference, but only one of the four connection points can be seen in the side elevational view of FIG. 1A. FIG. 1B is a schematic illustration of two of the circumferential loops 110 separated from each other. Other embodiments of stents may have sawtoothed shaped circumferential loops attached to each other at more points around the circumference. FIG. 1C, for example, shows a schematic side elevational view of a stent 150 in which the sawtooth shaped circumferential loops are attached to each other at every sawtooth apex.

It should be apparent from the above description of the stents depicted in FIGS. 1A and 1C that one can trace many different closed loop conducting paths in either of those stents. For example, a circular closed loop conducting path may be traced around each sawtoothed shaped circumferential loop 10. It should also be apparent that one could trace longitudinal conducting paths in either stent 100 in FIG. 1A or stent 150 in FIG. 1C by moving from one circumferential loop 110 to the next circumferential 110 through the connection points 120 in the same longitudinal row. In stent 100 in FIG. 1A there are four such longitudinal conducting paths along the four rows of connection points spaced at 90° intervals around the circumference of stent 100. In stent 150 in FIG. 1C there could be many such longitudinal conducting paths. Furthermore, in stent 100 in FIG. 1A or stent 150 in FIG. 1C, one may trace helical conducting paths by moving from one circumferential loop 110 to the next circumferential loop 110 at connecting points in successively different longitudinal rows.

While stents as illustrated in FIGS. 1A and 1C are common, and are disclosed embodiments in this specification, the invention is not limited to stents comprising connected sawtooth shaped circumferential loops. The invention may be applied to any tubular stent in which closed loop conducting loops can be traced.

When either of stents 100 or 150 is implanted in a subject and placed in a MRI field, the varying magnetic field of the MRI gradient and radio frequency imaging radiation will induce currents in the conducting tubular mesh structure of stent 100 or 150. As described above, many closed loop conducting paths exist in stent 100 or 150 in which such induced currents could flow. Such induced currents produce, via Lenz's law, varying magnetic fields that oppose the varying magnetic fields of the incident RF radiation, thereby distorting and/or reducing the contrast of the resulting magnetic resonance image. For the sake of simplicity, in the following detailed description of the invention, the embodiments will be described in terms of coatings disposed on a single conducting circular ring. The single conducting circular ring will serve as a surrogate for any of the closed loop conducting paths in stents 100 or 150 as described above.

While the embodiments will be described in terms of coatings disposed on a single conducting circular ring, it will be obvious to those of ordinary skill in the art that such embodiments can be extended to the structure of stent 100 or 150 in FIGS. 1A and 1C respectively. Additionally, it should be obvious to those of ordinary skill in the art that embodiments described in terms of coatings disposed on a single conducting circular ring may also be extended to any situation in which electromagnetic radiation is incident on any device comprised of a conducting substrate with one or more holes therein. The perimeter of each such hole is the analog of a single conducting circular ring.

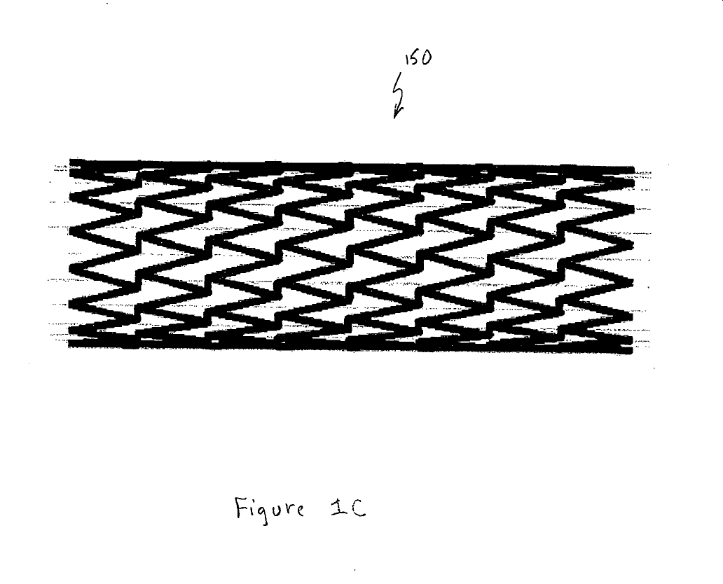

One embodiment is depicted schematically in FIG. 2. Referring to FIG. 2, there is shown a cross-sectional view of a coated ring assembly 200 comprising a conducting ring 210 coated with a plurality of coated layers 220, 230, 240, and 250. Conducting ring 210 is first completely coated with a first electrically insulating layer 220. First insulating layer 220 is then coated with a first electrically conducting layer 230. First conducting layer 230 is not coated on the entire circumference of conducting ring 210, but rather has an angular gap β. Angular gap β is sufficient to completely break the otherwise continuous circumferential conductive path of conducting layer 230 around the ring 210. In one embodiment angular gap β is in the range from about 0.50 to 5.00. A second insulating layer 240 is then coated over first conducting layer 230 for the entire circumference of conducting ring 210. In one embodiment the insulating layer 240 also fills in the angular gap β formed in conducting layer 230. In another embodiment, not shown, the material deposited in the angular gap β, formed in conducting layer 230, is a different insulating and/or dielectric material than the insulating material 240. A second conducting layer 250 is then coated over second insulating layer 240. Second conducting layer 250 is coated on only an angular portion α of the circumference of conducting ring 210 centered over angular gap β in first conducting layer 230. In one embodiment angular portion α may be about 20°. In another embodiment the angular portion a may be about 180°. Angular arc lengths α and β in FIG. 2 may alternatively be expressed as percentages of a complete 360° or continuous coating. This alternative way of expressing the arc lengths α and β is particularly appropriate in other embodiments of the invention applied to conducting loops that are not circular or even curvilinear, i.e., conducting loops comprising connected linear segments.

When coated ring assembly 200 is placed in a MRI field, the RF imaging radiation of the MRI field will induce currents in conducting ring 210 and in conducting layers 230 and 250. As discussed above, such induced currents produce induced RF magnetic fields that oppose the incident MRI RF magnetic fields that produced the induced currents and, as a result, distort or even obliterate the MR images. Applicants have discovered that the phase and amplitude relationship between the currents induced in ring 210 and the currents induced in layers 230 and 250 depends upon several properties and parameters of layers 220, 230, 240, and 250. Without wishing to be bound by any particular theory, it is believed that layers 220, 230, 240, and 250 may be modeled as an equivalent, inductively coupled, RLC circuit driven by the incident RF imaging radiation of the MRI field. The equivalent values of R, L, and C will determine the phase and amplitude relationship between the currents induced on layers 230, 240, and 250 and the current induced in the ring 210.

Referring again to FIG. 2, in one embodiment it is desired that the current induced in the combination of layers 230, 240, and 250 be nearly in phase with, and nearly the same amplitude as, the current induced in the ring 210. In another embodiment it is desired that the current induced in the combination of layers 230, 240, and 250 be out of phase and differ in amplitude, by predetermined amounts, with the current induced in the ring 210. The phase and amplitude relationship between the currents induced in the combination of layers 230, 240, and 250 and the current induced in the ring 210 depends upon the relationship of the frequency of the RF imaging radiation to the resonant frequency of the equivalent RLC circuit of the coated ring assembly 200. As already described above, the currents induced in the coated ring assembly 200 will, in turn, create induced magnetic fields that oppose the magnetic fields that created the induced currents, namely the radio frequency magnetic fields of the MRI field. The phase and amplitude relationship of the induced magnetic fields to the incident MRI magnetic fields will therefore be directly related to the phase and amplitude relationship of the currents induced in coated ring assembly 200 to the currents induced in the uncoated ring 210.

For a description of resonant circuits reference may be had, e.g., to Chapter 19, beginning at page 675, of J. Richard Johnson's “Electric Circuits” (Hayden Book Company, Hasbrouck Heights, N.J., 1984). Reference may also be had to TheFreeDictionary.com by Farlex which may be found at the Internet web site www.encyclopedia.thefreedictionary.com/RLC%20circuit and which states:

“In an electrical circuit, resonance occurs at a particular frequency when the inductive reactance and the capacitive reactance are of equal magnitude, causing electrical energy to oscillate between the magnetic field of the inductor and the electric field of the capacitor.

Resonance occurs because the collapsing magnetic field of the inductor generates an electric current in its windings that charges the capacitor and the discharging capacitor provides an electric current that builds the magnetic field in the inductor, and the process is repeated. An analogy is a mechanical pendulum.

At resonance, the series impedance of the two elements is at a minimum and the parallel impedance is a maximum. Resonance is used for tuning and filtering, because resonance occurs at a particular frequency for given values of inductance and capacitance. Resonance can be detrimental to the operation of communications circuits by causing unwanted sustained and transient oscillations that may cause noise, signal distortion, and damage to circuit elements.

Since the inductive reactance and the capacitive reactance are of equal magnitude, ωL=1/ωC, where ω=2πf, in which f is the resonant frequency in hertz, L is the inductance in henries, and C is the capacitance in farads when standard SI units are used.”

TheFreeDictionary.com goes on to state: “The Q factor or quality factor is a measure of the “quality” of a resonant system. Resonant systems respond to frequencies close to the natural frequency much more strongly than they respond to other frequencies.

On a graph of response versus frequency, the bandwidth is defined as the part of the frequency response that lies within 3 dB about the center frequency. The definition of the bandwidth as the “Full Width at Half Maximum” or FWHM is wrong.

The Q factor is defined as the resonant frequency (center frequency f₀) divided by the bandwidth Δf or BW:

$$Q = \frac{{f}_{0}}{{f}_{2} - {f}_{1}} = \frac{{f}_{0}}{Δ f}$$

Bandwidth BW or Δf=f₂−f₁, where f₂ is the upper and f₁ the lower cutoff frequency. In a tuned radio frequency receiver (TRF) the Q factor is:

$$Q = \frac{1}{R} \sqrt{\frac{L}{C}}$$

where R, L, and C are the resistance, and capacitance of the tuned circuit, respectively.”

TheFreeDictionary.com further states: “An RLC circuit is a kind of electrical circuit composed of a resistor (R), an inductor (L), and a capacitor (C). See RC circuit for the simpler case. A voltage source is also implied. It is called a second-order circuit or second-order filter as any voltage or current in the circuit is the solution to a second-order differential equation.
The resonant or center frequency of such a circuit (in hertz) is:

$${f}_{c} = \frac{1}{2 π \sqrt{LC}}$$

It is a form of bandpass or bandcut filter, and the Q factor is

$$Q = \frac{{f}_{c}}{BW} = \frac{2 π {f}_{c} L}{R} = \frac{1}{\sqrt{{R}^{2} C / L}}$$

Reference may also be had to U.S. Pat. No. 6,667,674 (“NMR resonators optimized for high Q factor”), the entire disclosure of which is hereby incorporated by reference into this specification.

Referring again to FIG. 2, if the current induced in the combination of layers 230, 240, and 250 is about 180° out of phase with, and about equal in magnitude to, the current induced in the ring 210, those two induced currents will destructively interfere, thereby resulting in the electromagnetic fields inside the ring being nearly the same as if the ring were not present. Such a result would mean that the presence of the ring had distorted the resulting MR image only a small amount, and thereby enable MR imaging of material within the ring.

The magnetic fields resulting from placing coated ring assembly 200 into an electromagnetic radiation field were analyzed using Ansoft Maxwell 3D magnetic field finite element analysis software. The modeled ring was a conducting ring with a 6 mm I.D., 1 mm long, and with a 0.25 mm wall thickness. Finite element simulations were first run for an uncoated ring to determine the magnetic field strength at the center of the ring and the induced current in the ring. Coated layers, as per FIG. 2, were then added to the ring and the simulations run again. From these simulations, the inductance and resistance of the coatings were determined.

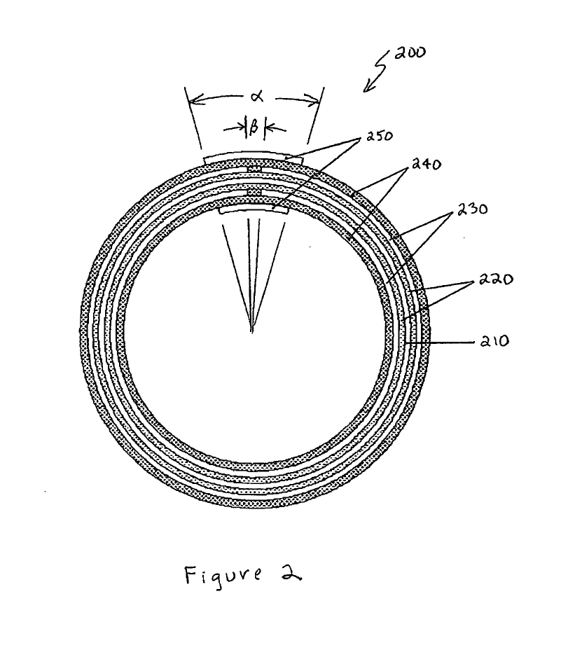

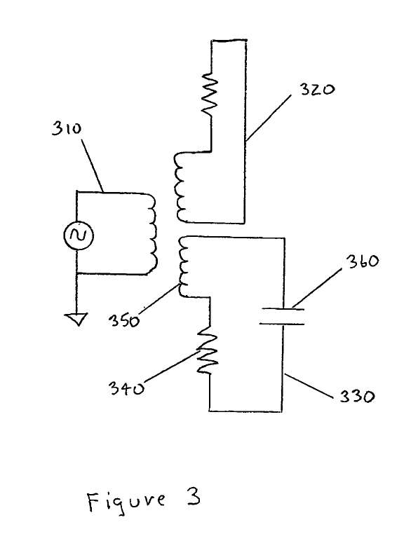

Equivalent circuits were then proposed in which the RF transmitter, copper ring, and coated layers were modeled as three separate circuits inductively coupled to each other. FIG. 3 illustrates the modeled circuits. In FIG. 3 circuit 310 represents the RF transmitter of the MRI machine, circuit 320 represents the copper ring, and circuit 330 represents the coated layers. The resistance and inductance circuit parameter values for coated layer resistor 340 and inductor 350, respectively, were taken from the finite element field simulations. A series capacitor 360 was inserted into the coated layer circuit 330 and circuit simulations were run to determine the capacitor size that would achieve the desired result, in this case an approximate 180° phase difference between the induced current in the ring and the induced current in the coated layers and with their current amplitudes approximately equal. Exact matches could not be obtained (180° phase difference and exact matching amplitudes). However, good results were possible with a phase difference of approximately 170°, with a capacitance value for capacitor 360 of between 9 and 10 nanofarads. FIG. 4 is a plot, from the simulation, of the induced currents in the ring 210 (curve 410), in the coated layers (curve 420), and the sum of the induced current in the ring and in the coated layers (curve 430).

The simulation results described above were obtained by iteratively varying the parameters of the coated layers in coated ring assembly 200 of FIG. 2, namely the resistance of conducting layers 230 and 250, the dielectric constant and thickness of insulating layers 220 and 240, and the extent of angular portion α, until the result depicted in FIG. 4 were achieved. The resistance of conducting layers 230 and 250 is varied by the selection of conducting material (varying the conductivity) and the thickness of the layers (varying the conductance). The dielectric constant of insulating layers 220 and 240 is varied by varying the relative permittivity of the simulated material. Varying the thickness of insulating layers 220 and 240, and the relative permittivity values the simulated materials, varies the capacitance of the capacitor 360 in the equivalent circuit model shown in FIG. 3. In one simulation, the results graphed in FIG. 4 required a large dielectric constant of 1395 with a thickness of insulating layers 220 and 240 of 2.0 μm and an angular portion α of 90°. However by reducing the thickness of insulating layers 220 and 240 to 0.2 μm, the required dielectric constant was 139.

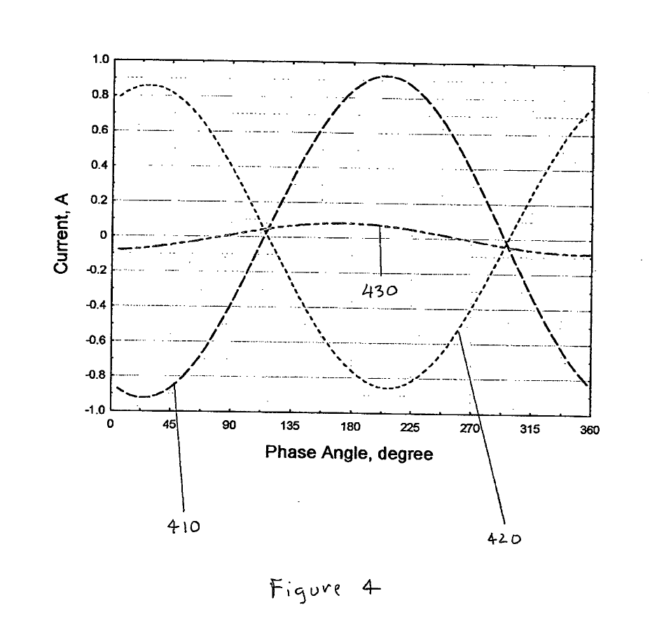

Using variations of the geometry of the coated layers to increase the capacitance may further reduce the required dielectric constant. FIG. 5 is a schematic cross-sectional view of another embodiment with a coating geometry capable of producing a higher capacitance. Referring to FIG. 5, a conducting ring 510 is first completely coated with a first insulating layer 520. First insulating layer 520 is then coated with a first electrically conducting layer 530. First conducting layer 530 is coated around the circumference of conducting ring 510, in a spiral fashion, so that it overlaps itself by an angular amount a as it completes more than 360° around the circumference of conducting ring 510. A second insulating layer 540 is coated over first conducting layer 530 in the overlap portion α, thus preventing electrical contact of conductive coating 530 with itself in the region of overlap. In one embodiment angular overlap portion a may be anywhere in the range from about 1° to about 360°. In another embodiment angular overlap portion a may extend beyond 360° thereby encircling conducting ring 510 multiple times and creating multiple regions of overlap. FIG. 5A is a schematic cross-sectional view a coated ring assembly 550, similar to coated ring assembly 500 in FIG. 5, but with angle α being about 360°. For the same thickness of second insulating layer 240 in FIGS. 2 and 540 in FIG. 5, the coating geometry of FIG. 5 yields a higher capacitance thereby requiring a lower dielectric constant. As previously discussed in reference to coated ring assembly 200 in FIG. 2, angular overlap portion a in FIG. 5 may alternatively be expressed as a percentage of a complete 360° coating. In this alternative a 360° overlap portion α would be expressed as a 100% overlap portion. A 720° overlap portion a would be expressed as a 200% overlap portion, etc. This alternative way of expressing the arc length α of coated ring assembly 500 in FIG. 5 is particularly appropriate in other embodiments of the invention applied to conducting loops that are not circular or even curvilinear, i.e., conducting loops comprising connected linear segments.

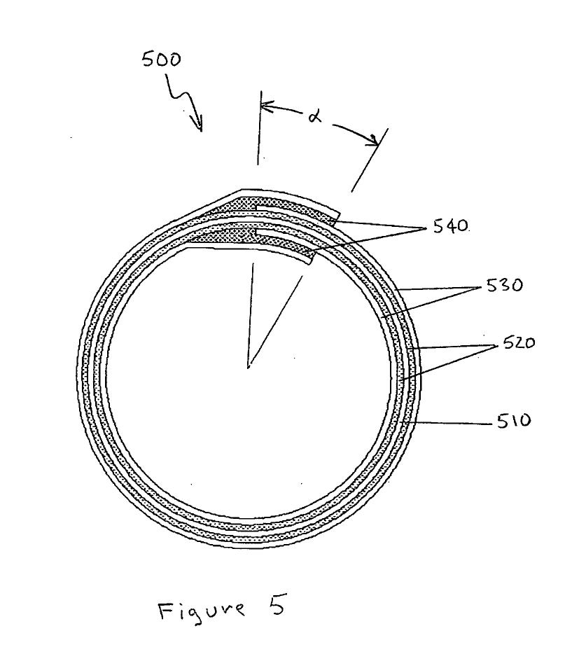

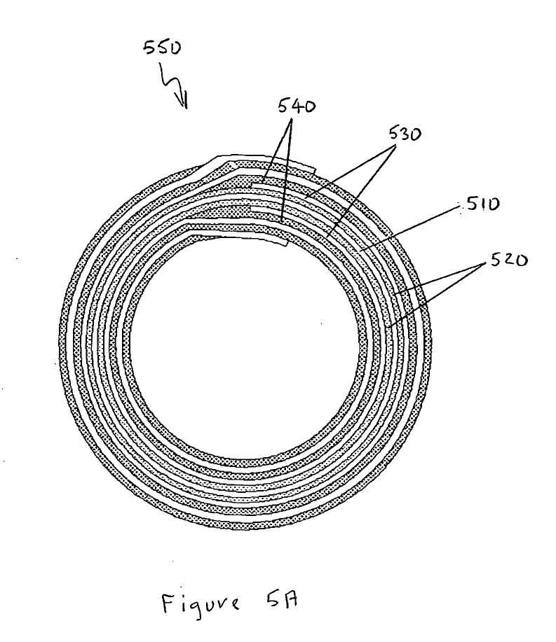

FIG. 6 is a schematic cross-sectional view of another embodiment, namely coated ring assembly 600. Coated ring assembly 600 is essentially coated ring assembly 500 of FIG. 5, but with two additional coated layers, third insulating layer 660 over second conducting layer 550 of FIG. 5, and third conducting layer 670 over third insulating layer 660. Third conducting layer 670 is electrically connected to first conducting layer 550 at point 690. Third insulating layer 660 and third conducting layer 670 are disposed over angular segment δ. In one embodiment, angular segment δ may be anywhere in the range from about 1° to about 90°. In another embodiment, angular segment 8 may be 360° or more, thereby creating a spiral coating with layers 660 and 670 similar to that of layers 530 and 540 in FIG. 5A.

In one embodiment, conducting layers 230 and 250 in FIG. 2, 530 in FIG. 5, and 670 in FIG. 6 preferably have an electrical conductivity greater than 3.0×10⁷ siemans/meter, a thickness in the range from 10 nanometer to 1 millimeter, and may be comprised of aluminum, gold, copper, silver, or other conductive materials and composites. Insulating layers 220 and 240 in FIGS. 2, 520 and 540 in FIG. 5, and 660 in FIG. 6 may have an electrical resistivity of at least 10⁵ ohm-centimeters, a dielectric constant in the range from 1.1 to 5000, a thickness in the range from 10 nanometer to 1 millimeter, and may be comprised of aluminum nitride, barium titanate, tantalum oxide, aluminum oxide, ceramics—typically alumina or aluminosilicates, glasses—typically borosilicate, polyesters, polyamides, SiO₂, Si₃N₄, Al₂O₃, Y₂O₃, La₂O₃, Ta₂O₅, TiO₂, HfO₂, ZrO₂, as well as composite mixes composed of dielectric materials with embedded conductive particles (see, for example, the materials described in the article “Controlling the Properties of Electromagnetic Composites” by P. S. Neelakanta, Advanced Materials & Processes, Vol. 3, 1992, pp. 20-25).

Conducting layers 230 and 250 in FIG. 2, 530 in FIGS. 5 and 5A, and 670 in FIG. 6 may be deposited by any of several coating techniques known to those skilled in the art, for example, evaporative coating, sputtering, or chemical vapor deposition. Conventional sputtering techniques, for example, may be referenced in U.S. Pat. No. 5,835,273 (“Deposition of an aluminum mirror”); U.S. Pat. No. 5,711,858 (“Deposition of aluminum alloy film”); and U.S. Pat. No. 4,976,839 (“Aluminum electrode”). The entire disclosure of each of these United States patents is hereby incorporated by reference into this specification. Insulating layers 220 and 240 in FIGS. 2, 520 and 540 in FIG. 5, and 660 in FIG. 6 may also be deposited by the above techniques or, depending upon the material, by conventional solvent coating or spraying techniques. To achieve the geometrical coating patterns on coated ring assembly 200 in FIG. 2, 500 in FIG. 5, and 600 in FIG. 6 conventional masking techniques known to those skilled in the art, for example photo-etching, may be employed. Reference may be had, for example, to U.S. Pat. Nos. 5,851,364; 5,685,960; 6,222,271; and 6,194,783. The entire disclosure of each of these United States patents is hereby incorporated by reference into this specification.

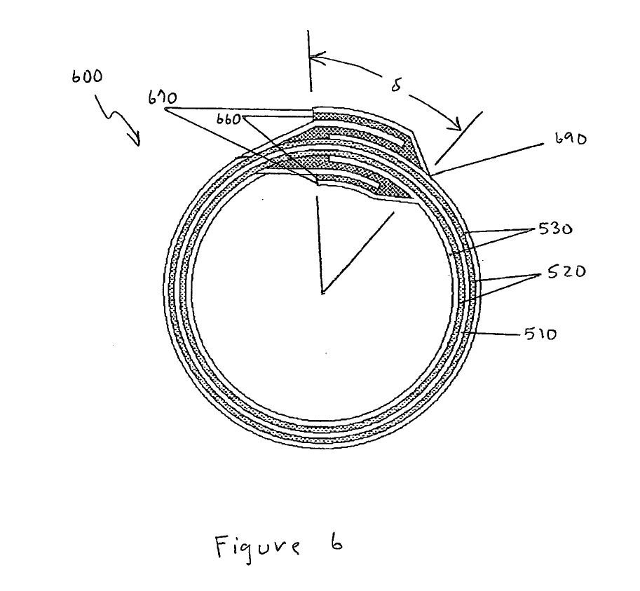

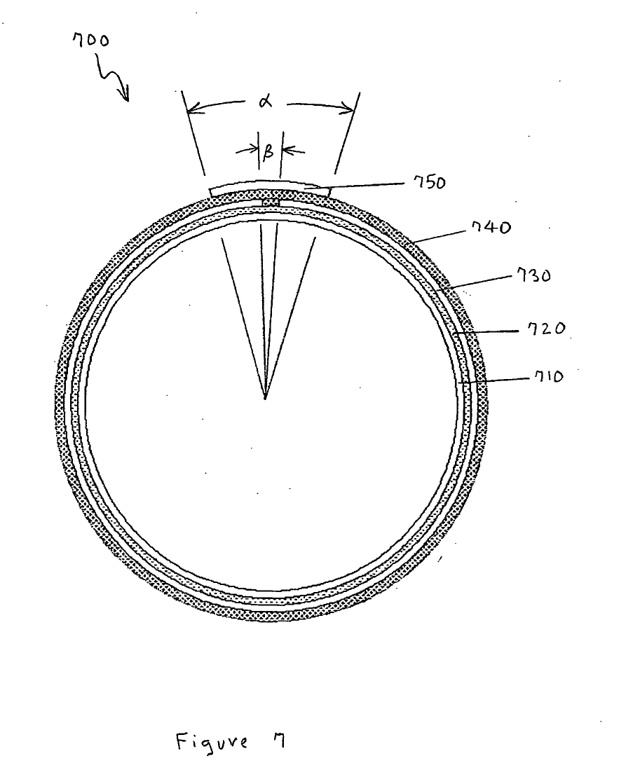

The coated ring assembly embodiments 200 in FIG. 2, 500 in FIG. 5, and 600 in FIG. 6 have the coated layers disposed completely around the smaller diameter of the ring annular surface. In another embodiment the coated layers are coated on only a portion of the annular surface of the ring. FIG. 7 illustrates a coated ring assembly 700 similar to the embodiment in FIG. 2 but with each layer coated on only the outer surface of conducting ring 710. The outer surface of conducting ring 710 is first coated with a first electrically insulating layer 720. First insulating layer 720 is then coated with a first electrically conducting layer 730. First conducting layer 730 is not coated on the entire outer circumference of conducting ring 710, but rather has an angular gap β. Angular gap β is sufficient to completely break the otherwise continuous circumferential conductive path of conducting layer 730 around the ring 710. In one embodiment angular gap β is in the range from about 0.5° to 5.0°. A second insulating layer 240 is then coated over first conducting layer 730 for the entire outer circumference of conducting ring 710. In one embodiment the insulating layer 740 also fills in the angular gap β formed in conducting layer 730. In another embodiment, not shown, the material deposited in the angular gap β, formed in conducting layer 730, is a different insulating and/or dielectric material than the insulating material 740. A second conducting layer 750 is then coated over second insulating layer 740. Second conducting layer 750 is coated on only an angular portion α of the outer circumference of conducting ring 710 centered over angular gap β in first conducting layer 730. In one embodiment angular portion α may about 20°. In another embodiment the angular portion α may be in the range from about 20° to about 180°. Angular arc lengths α and β in FIG. 8 may alternatively be expressed as percentages of a complete 360° or continuous coating. This alternative way of expressing the arc lengths α and β is particularly appropriate in other embodiments of the invention applied to conducting loops that are not circular or even curvilinear, i.e., conducting loops comprising connected linear segments.

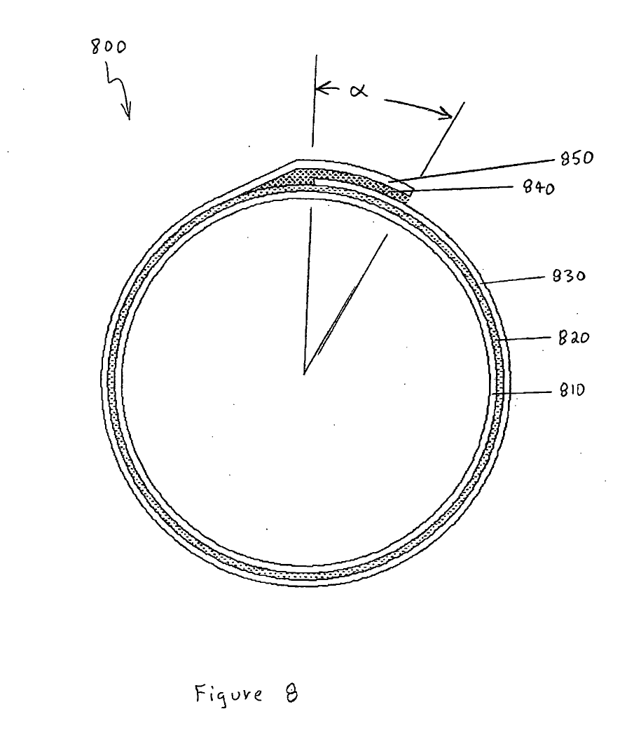

FIG. 8 is a schematic cross-sectional view of another embodiment, namely coated ring assembly 800. Coated ring assembly 800 is similar to the embodiment in FIG. 5, but with each layer coated on only the outer surface of conducting ring 810. Referring to FIG. 8, the outer surface of conducting ring 810 is first coated with a first insulating layer 820. First insulating layer 820 is then coated with a first electrically conducting layer 830. First conducting layer 830 is coated on the entire outer circumference of conducting ring 810, in a spiral fashion, so that it overlaps itself by an angular amount a as it completes more than 360° around the circumference of conducting ring 810. A second insulating layer 840 is coated over first conducting layer 830 in the overlap portion α, thus preventing electrical contact of conductive coating 830 with itself in the region of overlap. In one embodiment angular overlap portion a may be anywhere in the range from about 1° to about 360°. In another embodiment angular overlap portion a may extend beyond 360° thereby encircling conducting ring 510 multiple times and creating multiple regions of overlap. FIG. 8A is a schematic cross-sectional view a coated ring assembly 850, similar to coated ring assembly 800 in FIG. 9, but with angle α being greater than 360°. As previously discussed in reference to coated ring assembly 500 in FIG. 5, angular overlap portion α in FIG. 8 may alternatively be expressed as a percentage of a complete 360° coating. In this alternative a 360° overlap portion α would be expressed as a 100% overlap portion. A 720° overlap portion α would be expressed as a 200% overlap portion, etc. This alternative way of expressing the arc lengths α of coated ring assembly 800 in FIG. 8 is particularly appropriate in other embodiments of the invention applied to conducting loops that are not circular or even curvilinear, i.e., conducting loops comprising connected linear segments.

FIG. 9 is a schematic cross-sectional view of another embodiment, namely coated ring assembly 900. Coated ring assembly 900 is similar to the embodiment in FIG. 6, but with each layer coated on only the outer surface of conducting ring 910. Coated ring assembly 900 is essentially coated ring assembly 800 of FIG. 8, but with two additional coated layers, third insulating layer 960 over second conducting layer 830 of FIG. 8, and third conducting layer 970 over third insulating layer 960. Third conducting layer 970 is electrically connected to first conducting layer 930 at point 990. Third insulating layer 960 and third conducting layer 970 are disposed over angular segment δ. In one embodiment, angular segment δ may be anywhere in the range from about 1° to about 90°.

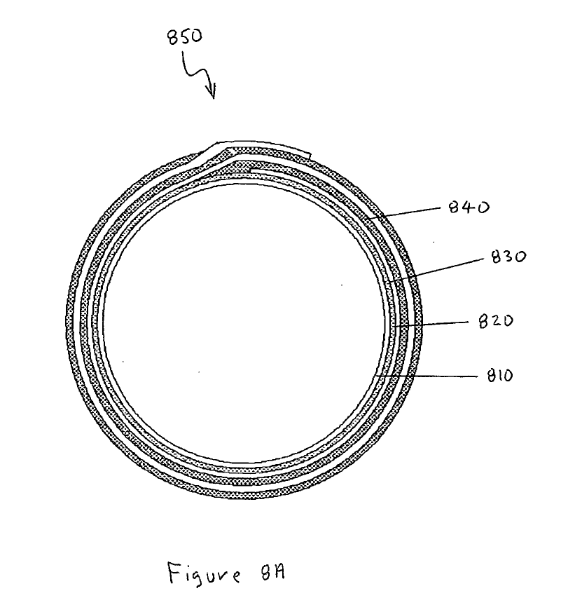

As previously discussed, the coated ring assembly embodiments disclosed above in FIGS. 2, 5, 6, 7, 8, and 9 are in terms of simple single coated conducting rings so as to simplify the drawings for the detailed description of the coated layer embodiments therein. Referring again to FIGS. 1A, 1B, and IC, any of the coating embodiments depicted in FIGS. 2, 5, 6, 7, 8, and 9 may be coated on one or more of the sawtoothed shaped circumferential loops 110 of stents 100 and 150. Any of the coated layer embodiments depicted in FIGS. 2, 5, 6, 7, 8, and 9 may also be applied to any of the closed loop conducting paths of stents 100 and 150 as has been discussed elsewhere in this specification. FIG. 10 depicts five possible closed loop conducting paths 160, 162, 164, 166, and 168 that may be coated with the coated layer embodiments depicted in FIGS. 2, 5, 6, 7, 8, and 9. For the sake of simplicity, the closed loop conducting paths in FIGS. 10A-E are depicted as smooth lines, but it must be understood that they would be jagged lines in stents 100 or 150 of FIGS. 1A and 1C respectively.

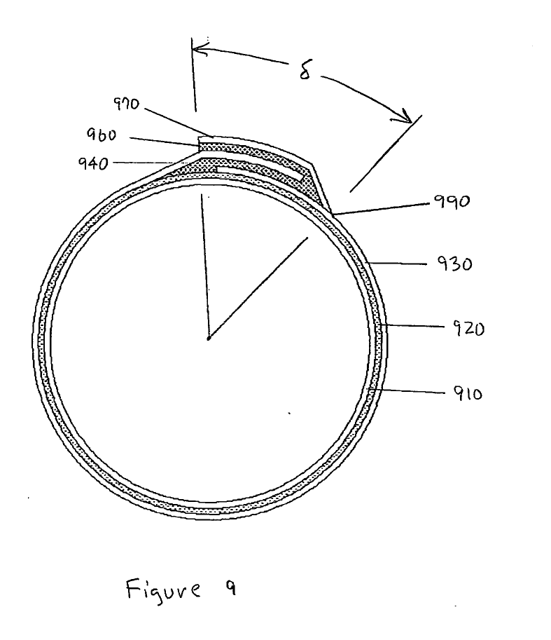

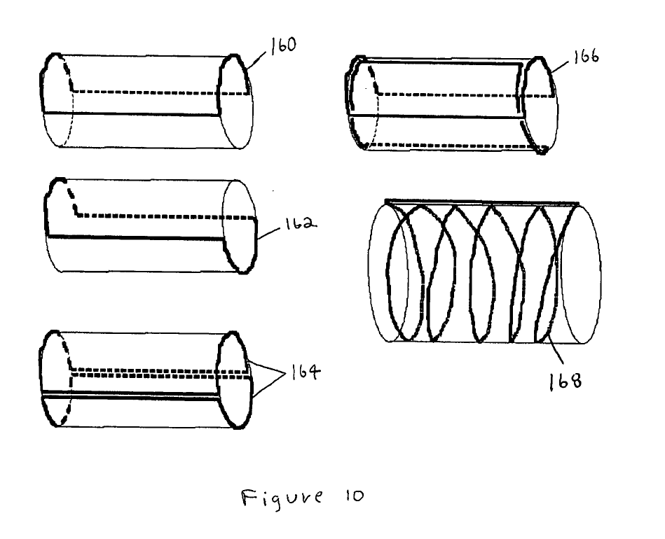

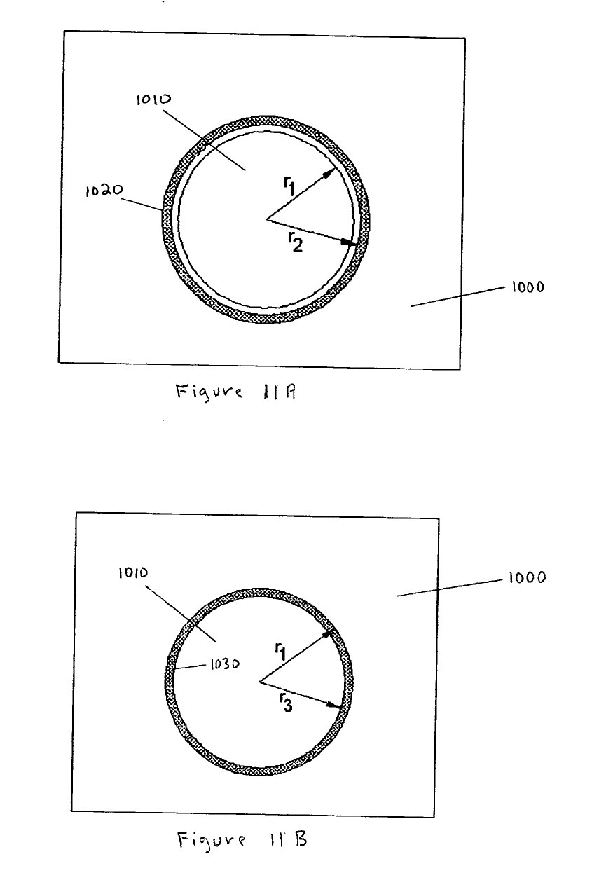

Additionally, it should be obvious to those skilled in the art that the coated layer embodiments described in terms of coatings disposed on a single conducting circular ring may also be extended to any device comprised of a conducting substrate with one or more holes therein, wherein electromagnetic radiation is incident on the device. The perimeter of each such hole is the analog of a single conducting circular ring. FIGS. 11A and 11B illustrate a portion of a conducting plate 1000 with a hole 1010 of radius r₁ therein. Any of the coated layer patterns illustrated in FIGS. 7, 8, and 9 may be disposed around the perimeter of hole 1010, either on the outer surface of the plate 1000 as is illustrated in FIG. 11A, or on the inner surface of the hole 1010 as is illustrated in FIG. 11B. In FIG. 11A the shaded area 1020 of radius r₂ represents the plurality of stacked coated layers of either FIG. 7, 8, or 9 on the outer surface of plate 1000. In FIG. 11B the shaded area 1030 of radius r₃ represents the plurality of stacked coated layers of either FIG. 7, 8, or 9 on the inner surface of hole 1010.

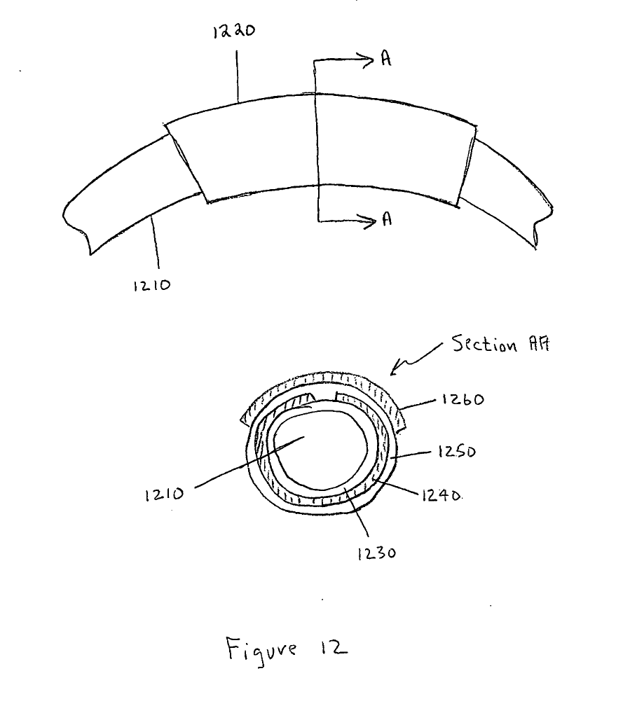

Referring to FIG. 12, in another embodiment coated layer assemblies similar to the coated layer assemblies depicted in FIGS. 7, 8, 8A, and 9, instead of being coated around the large diameter of the rings (or any of the closed loop conducting paths of a stent), are coated around sections of the annular body of the rings (or around sections of the framework of a stent). Referring again to FIG. 12, there is depicted a section 1210 of the framework of a stent. Coated around a portion of section 1210 is a coated layer assembly 1220. AA is a cross-sectional view of coated layer assembly 1220. In the cross-sectional view AA is shown section 1210 coated with a first insulating layer 1230, a first conducting layer 1240, a second insulating layer 1250, and a second conducting layer 1260. As is apparent, the coated layer assembly depicted in Section AA of FIG. 12 is similar to the assembly of layers coated around the outer annular surface of ring 710 in FIG. 7. In other embodiments, not shown, the layer assemblies coated around the outer annular surfaces of rings 810 in FIGS. 8 and 8A and around the outer annular surface of ring 910 in FIG. 9 are coated around section 1210 in FIG. 12 instead of the layer assembly shown in section AA in FIG. 12.

Some of the conducting materials that may be used for the top-most conducting layers in all of the coated layer embodiments disclosed above in this specification may be incompatible with the biological tissues in which the coated devices are implanted. If the top-most conducting layer is incompatible with the biological tissue in which the coated device is implanted, the device will be coated with a final insulating layer, which isolates the top-most conducting layer from the biological tissue in which the device is implanted. Such a final coated layer is not shown in any of the figures of embodiments as described above, but it should be understood that those embodiments will additionally comprise such a final coated layer when required for compatibility of the implanted device with the surrounding biological tissue. Such a final insulating coated layer will not affect the advantageous affect of the underlying coated layers.

Determination of Resonant Frequency

As is known to those skilled in the art, the electrical characteristics of an electrical circuit can change depending on the environment into which the circuit is placed. For example, parasitic capacitance can form at the interface of the circuit's materials and the circuit's environment. Hence, the response, and in particular a resonance response, of a circuit or a system comprising a circuit depends on the environment into which the system is placed. Thus, a system which resonates at one frequency in an air environment may resonate at a different frequency in an essentially liquid and/or semi-liquid environment of a patient's body.

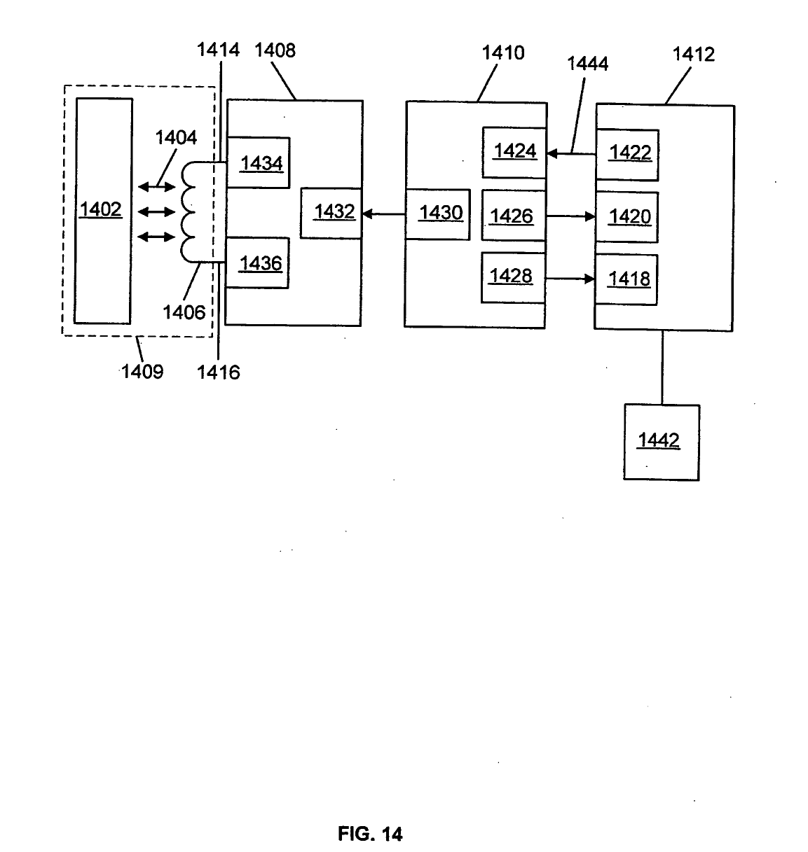

In the process of this invention, certain resonance characteristics are achieved by the stent system. In one embodiment, the stent system comprises a vascular stent. In another embodiment the stent system comprises a vascular stent and an electrical circuit in the proximity of and/or in contact with a portion of the vascular stent. In another embodiment, the stent system comprises a vascular stent, an electrical circuit in the proximity of and/or in contact with a portion of the vascular stent, and the tissue and fluids contained within and around the vascular stent when the stent is positioned into a patient. In another embodiment, the stent system comprises a vascular stent, an electrical circuit in the proximity of and/or in contact with a portion of the vascular stent, and substitute materials which can be substituted for the patient's tissues and fluids and have essentially the same electrical and magnetic properties as said patient's tissues and fluids. In another embodiment, the stent system comprises a vascular stent, an electrical circuit in the proximity of and/or in contact with a portion of the vascular stent, substitute materials which can be substituted for the patient's tissues and fluids and have essentially the same electrical and magnetic properties as the said patient's tissues and fluids, and a container to contain said stent, electrical circuit and substitute materials within a measurement system, e.g., as depicted in FIG. 14. In one embodiment, the container of the stent system is comprised of a glass beaker. In another embodiment, the container of the stent system is a Pyrex container. In yet another embodiment, the container of the stent system is comprised of a polymer material, e.g., a plastic, a nylon or the like. In one embodiment, said container is a nonconductive and nonmagnetic container suitable for containing liquids at essentially room temperature.

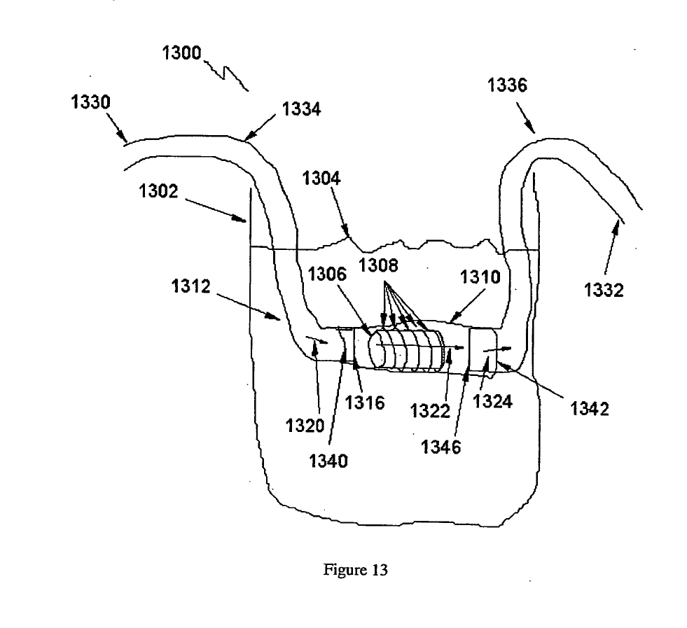

FIG. 13 depicts one embodiment of a stent system 1300 comprising a vascular stent 1306 submerged in a material 1304 contained in a container 1302. The container 1302 may be, e.g., a glass beaker, a plastic container or other non-electrically conductive and nonmagnetic container suitable for containing material 1304 in a room temperature environment. Material 1304 may be, e.g., a liquid material, a gelled material or the like. In one embodiment, material 1304 may be blood. In another embodiment material 1304 may be a material with essentially the same electrical and magnetic properties of muscle tissue.

Continuing to refer to FIG. 13 and to the embodiment depicted therein, stent 1306 is in the proximity of an RLC circuit 1308 which may be, e.g. one of the circuit configurations disclosed in this application. The stent 1306 and RLC circuit 1308 is positioned within a tubular material 1310. Material 1310 may be, e.g. a portion of an animal artery, or other vascular material, or a vascular substitute which has essentially the same electromagnetic properties of human vascular tissue. Material 1310 is attached to tubes 1334 and 1336. Material 1310 has an end 1340 attached to the end 1316 of tubing 1334. Material 1310 has an end 1342 attached to the end 1346 of tubing 1336. A pump (not shown and not part of the stent system) pumps a liquid 1320, 1322, 1342, through the tubing 1330, through the material 1310 and through the tubing 1336. Said liquid may be, e.g., blood or other liquid which has essentially the same electric and magnetic properties of blood. The moving liquid 1320 passes though the tubing 1334 and enters the material 1310 to become the moving liquid 1322 which also passes through the stent 1306. Liquid 1322 passes through the material 1310 to exit the material 1310 as moving liquid 1324 and enters the tubing 1336 at tub end 1346.

The pump (not shown and not part of the stent system) may pulse the flow of liquids 1320, 1322, 1324 to simulate essentially the pulse flow of blood in a body.

The resonance characteristics of the said stent system may be determined by the test method depicted in FIG. 14 or by other conventional means known to those skilled in the art.

FIG. 14 depicts one embodiment of an impedance test apparatus suitable for determining the resonance frequency of the stent system. An Agilent Technologies, Inc. model 4395A-010 network/spectrum/impedance analyzer 1412 comprises a display and is operationally connected to an Agilent Technologies, Inc. model 43961A test impedance kit 1410 which is operationally connected to an Agilent Technologies, Inc. model 16092A test fixture 1408. Additionally and optionally an Agilent Technologies, Inc. model 85032E calibration kit 1442 may be connected to the said network/spectrum/impedance analyzer 1412 and, as is known to those skilled in the art, may be used to calibrate said Agilent Technologies, Inc. model 4395A-010 network/spectrum/impedance analyzer 1412 before a measurement is performed.

In the embodiment depicted, said Agilent Technologies, Inc. model 4395A-010 Network/spectrum/impedance analyzer 1412 RF output port 1422 is operationally connected to said Agilent Technologies, Inc. model 43961A test impedance kit 1410 RF input port 1424 by an N-N cable 1444. Further, the R connections 1426, 1420 and A connections 1418, 1428 are appropriately connected between said devices.

Said test impedance kit 1410 is operationally connected to said test fixture 1408 at the output port 1430 of said test impedance kit 1410 and port 1432 of the test fixture 1408.

A single wire wound measurement solenoid coil 1409 which operationally is an inductor 1406 comprises leads 1414 and 1416 (which are the two ends of the wire used to construct the measurement solenoid coil 1409) surrounds the stent system 1402 under test. Said leads 1414 and 1416 are electrically connected to ports 1434, 1436 of said test fixture 1408. Thus, as is known to those skilled in the art, a single port connection is operationally made to the Network/spectrum/impedance analyzer 1412.

The stent system 1402 under test inductively couples 1404 to the measurement solenoid 1409 which operationally acts as an inductor 1406, thus, and as is known to those skilled in the art, changing the impedance characteristics of the measurement solenoid coil 1409 as a function of frequency.

As is known to those skilled in the art, the radio frequency signal produced by the Agilent Technologies, Inc. model 4395A-010 network/spectrum/impedance analyzer 1412 may be set to sweep from a frequency range of about 20 megahertz to about 100 megahertz, or about 40 megahertz to about 80 megahertz, or about 10 megahertz to about 300 megahertz, or about 100 kilohertz to about 500 megahertz.

As is known to those skilled in the art, the impedance of an electrical system is in general a complex number value and may be represented as
Z=R+iX

Where R is the resistance, X is the reactance and i is the square root of negative 1. As is known to those skilled in the art, the complex number part X of the impedance Z of the measurement solenoid 1409 around stent system 1402 is in part a function of frequency and can be graphed by the Agilent Technologies, Inc. model 4395A-010 network/spectrum/impedance analyzer 1412 as a function of the swept frequency range specified such that along the x-axis is the frequency and along the y-axis is the reactance X of the impedance measured.

As is known to those skilled in the art, the Agilent Technologies, Inc. model 4395A-010 network/spectrum/impedance analyzer 1412 directly measures impedance parameters operating in the radio frequency range of about 100 kilohertz to about 500 megahertz and with about a 3% impedance accuracy. The source level is from about −0.56 decibels per milliwatt to about +9 decibels per milliwatt at device under test and a direct current bias of about 40 volt and a maximum of about 20 milliamp and open/short/load compensation.

As is known to those skilled in the art, when the graphed reactance X crosses the x-axis a resonance condition is indicated having a frequency at the corresponding crossing point along the x-axis value.

In another embodiment, the Agilent Technologies, Inc. model 4395A-010 network/spectrum/impedance analyzer 1412 graphs the magnitude of the impedance |Z| as a function of frequency. The frequency is again along the x-axis. The magnitude of the impedance |Z| is along the y-axis. In this embodiment the resonance frequency of the stent system 1300 is the frequency at which |Z| is a maximum in the frequency range selected. It is to be understood that in any electrical system there may occur more than one resonance.

It is expressly understood that while the above discussion sets forth some preferred embodiments for implementing the invention and determining the resonance frequency, along with preferred frequency ranges of operation and apparatus configuration, any suitable implementation design could be constructed under the teachings herein and any suitable radio frequency transmission range or ranges could be used.

The foregoing description details the embodiments most preferred by the inventors. Variations to the foregoing embodiments will be readily apparent to those skilled in the relevant art. Therefore the scope of the invention should be measured by the appended claims.

We claim:

1. A device comprised of a plurality of surfaces, each said surface comprised of an electrically conductive material and having a plurality of apertures, each said aperture defined by a perimeter comprised of said conducting material; a plurality of layered coatings disposed on at least a portion of each said perimeter, said plurality of layered coatings being arranged so that radio frequency electromagnetic radiation, incident on said device, produces a first induced current in said conducting material and thereby a first induced magnetic field, and a second induced current in said layered coatings and thereby a second induced magnetic field, said second induced magnetic field having a predetermined phase and amplitude relationship to said first induced magnetic field.

2. The device as recited in claim 1, wherein said plurality of coated layers comprises a two-layer structure comprising a conducting layer over an insulating layer, said two-layer structure disposed around said perimeter in a spiral pattern with said insulating layer adjacent to said perimeter, thereby forming a plurality of overlapping segments of said two-layer structure around said perimeter.

3. The device recited in claim 2, wherein said insulating layer has a thickness of from about 1.0 nanometer to about 1.0 millimeter, and a dielectric constant of from about 1.1 to about 2000, and said conducting layer has a conductivity greater than 1.0×10⁶ siemans/meter, and a thickness of from about 1.0 nanometer to about 1.0 millimeter.

4. The device recited in claim 3, wherein said conducting layer is comprised of a material selected from the group consisting of aluminum, copper, gold, and silver.

5. The device as recited in claim 2, wherein said predetermined phase and amplitude relationship comprises a phase difference of from about 120° to about 180°, and an amplitude difference of from about 1% to about 100%.

6. The device as recited in claim 2, wherein said predetermined phase and amplitude relationship comprises a phase difference of from about 0° to about 20°, and an amplitude difference of from about 1% to about 100%.

7. The device recited in claim 1, wherein said plurality of layered coatings comprises a first insulating layer adjacent to said perimeter, a first conducting layer over said first insulating layer, a second insulating layer over said first conducting layer, and a second conducting layer over said second insulating layer.

8. The device recited in claim 7, wherein said first insulating layer is disposed continuously around said perimeter, said first conducting layer is disposed around about 90% of said perimeter, said second insulating layer is disposed continuously around said perimeter, and said second conducting layer is disposed around about 30% to about 90% of said perimeter.

9. The device recited in claim 8, wherein said first insulating layer has a resistivity greater than 10⁵ Ohm-centimeters, and a thickness of from about 1.0 nanometer to about 1.0 millimeter.

10. The device recited in claim 9, wherein said second insulating layer has a thickness of from about 1.0 nanometer to about 1.0 millimeter, and a dielectric constant of from about 1.1 to about 2000.

11. The device recited in claim 10, wherein said first conducting layer and said second conducting layer each have a conductivity greater than 1.0×10⁶ siemans/meter, and a thickness of from about 1.0 nanometer to about 1.0 millimeter.

12. The device recited in claim 11, wherein said first conducting layer and said second conducting layer each are comprised of a material selected from the group consisting of aluminum, copper, gold, and silver.

13. The device as recited in claim 8, wherein said predetermined phase and amplitude relationship comprises a phase difference of from about 120° to about 180°, and an amplitude difference of from about 1% to about 100%.

14. The device as recited in claim 8, wherein said predetermined phase and amplitude relationship comprises a phase difference of from about 0° to about 20°, and an amplitude difference of from about 1% to about 100%.

15. A stent for maintaining an open lumen in a duct in a living organism, said stent comprising a tubular skeletal structure comprised of an electrically conducting material and having a plurality of apertures defining a plurality of closed loop conducting paths in said electrically conducting material; a plurality of coated layers disposed on at least a portion of at least one of said closed loop conducting paths, said plurality of coated layers arranged so that radio frequency electromagnetic radiation, incident on said stent, produces a first induced current in said conducting material and thereby a first induced magnetic field, and a second induced current in said plurality of coated layers and thereby a second induced magnetic field, said second induced magnetic field having a predetermined phase and amplitude relationship to said first induced magnetic field.

16. The stent as recited in claim 15, wherein said plurality of coated layers comprises a two-layer structure comprising a conducting layer over an insulating layer, said two-layer structure disposed around said at least one of said closed loop conducting paths, in a spiral pattern, with said insulating layer adjacent to said tubular skeletal structure, thereby forming a plurality of overlapping segments of said two-layer structure.

17. The stent recited in claim 16, wherein said insulating layer has a thickness of from about 1.0 nanometer to about 1.0 millimeter, and a dielectric constant of from about 1.1 to about 2000; and said conducting layer has a conductivity greater than 1.0×10⁶ siemans/meter, and a thickness of from about 1.0 nanometer to about 1.0 millimeter.

18. The stent recited in claim 17, wherein said conducting layer is comprised of a material selected from the group consisting of aluminum, copper, gold, and silver.

19. The stent as recited in claim 16, wherein said predetermined phase and amplitude relationship comprises a phase difference of from about 120° to about 180°, and an amplitude difference of from about 1% to about 100%.

20. The stent as recited in claim 16, wherein said predetermined phase and amplitude relationship comprises a phase difference of from about 0° to about 20°, and an amplitude difference of from about 1% to about 100%.

21. The stent recited in claim 15, wherein said plurality of layered coatings comprises a first insulating layer adjacent to said at least one of said closed loop conducting paths, a first conducting layer over said first insulating layer, a second insulating layer over said first conducting layer, and a second conducting layer over said second insulating layer.

22. The stent recited in claim 21, wherein said first insulating layer is disposed continuously around the circumference of said at least one of said closed loop conducting paths, said first conducting layer is disposed around about 90% of the circumference of said at least one of said closed loop conducting paths, said second insulating layer is disposed continuously around the circumference of said at least one of said closed loop conducting paths, and said second conducting layer is disposed around the circumference of said at least one of said closed loop conducting paths in the range of about 30% to about 90%.

23. The stent recited in claim 22, wherein said first insulating layer has a resistivity greater than 10⁵ Ohm-centimeters, and a thickness of from about 1.0 nanometer to about 1.0 millimeter.

24. The stent recited in claim 23, wherein said second insulating layer has a thickness of from about 1.0 nanometer to about 1.0 millimeter, and a dielectric constant of from about 1.1 to about 2000.

25. The stent recited in claim 24, wherein said first conducting layer and said second conducting layer each have a conductivity greater than 1.0×10⁶ siemans/meter, and a thickness of from about 1.0 nanometer to about 1.0 millimeter.

26. The stent recited in claim 25, wherein said first conducting layer and said second conducting layer each are comprised of a material selected from the group consisting of aluminum, copper, gold, and silver.

27. The stent as recited in claim 22, wherein said predetermined phase and amplitude relationship comprises a phase difference of from about 120° to about 180°, and an amplitude difference of from about 1% to about 100%.

28. The stent as recited in claim 22, wherein said predetermined phase and amplitude relationship comprises a phase difference of from about 0° to about 20°, and an amplitude difference of from about 1% to about 100%.

29. A stent for maintaining an open lumen in a duct in a living organism, said stent comprising a tubular skeletal structure comprised of an electrically conducting material and having a plurality of apertures defining a plurality of closed loop conducting paths in said electrically conducting material; a plurality of coated layers disposed on at least a portion of at least one of said closed loop conducting paths, said plurality of coated layers arranged so that when an incident magnetic field of electromagnetic radiation is incident on said stent, an induced magnetic field at least as great as said incident magnetic field, is produced inside of said stent.

30. The stent as recited in claim 29, wherein said plurality of coated layers comprises a two-layer structure comprising a conducting layer over an insulating layer, said two-layer structure disposed around said at least one of said closed loop conducting paths, in a spiral pattern, with said insulating layer adjacent to said tubular skeletal structure, thereby forming a plurality of overlapping segments of said two-layer structure.

31. The stent recited in claim 30, wherein said insulating layer has a thickness of from about 1.0 nanometer to about 1.0 millimeter, and a dielectric constant of from about 1.1 to about 2000; and said conducting layer has a conductivity greater than 1.0×10⁶ siemans/meter, and a thickness of from about 1.0 nanometer to about 1.0 millimeter.

32. The stent recited in claim 31, wherein said conducting layer is comprised of a material selected from the group consisting of aluminum, copper, gold, and silver.

33. The stent recited in claim 29, wherein said plurality of layered coatings comprises a first insulating layer adjacent to said at least one of said closed loop conducting paths, a first conducting layer over said first insulating layer, a second insulating layer over said first conducting layer, and a second conducting layer over said second insulating layer.

34. The stent recited in claim 33, wherein said first insulating layer is disposed continuously around the circumference of said at least one of said closed loop conducting paths, said first conducting layer is disposed around about 90% of the circumference of said at least one of said closed loop conducting paths, said second insulating layer is disposed continuously around the circumference of said at least one of said closed loop conducting paths, and said second conducting layer is disposed around the circumference of said at least one of said closed loop conducting paths in the range of about 30% to about 90%.

35. The stent recited in claim 34, wherein said first insulating layer has a resistivity greater than 10⁵ Ohm-centimeters, and a thickness of from about 1.0 nanometer to about 1.0 millimeter.

36. The stent recited in claim 35, wherein said second insulating layer has a thickness of from about 1.0 nanometer to about 1.0 millimeter, and a dielectric constant of from about 1.1 to about 2000.

37. The stent recited in claim 36, wherein said first conducting layer and said second conducting layer each have a conductivity greater than 1.0×10⁶ siemans/meter, and a thickness of from about 1.0 nanometer to about 1.0 millimeter.

38. The stent recited in claim 37, wherein said first conducting layer and said second conducting layer each are comprised of a material selected from the group consisting of aluminum, copper, gold, and silver.

39. A stent for maintaining an open lumen in a duct in a living organism, said stent comprising a tubular skeletal structure comprised of an electrically conducting material and having a plurality of apertures defining a plurality of closed loop conducting paths in said electrically conducting material; a plurality of coated layers disposed on at least a portion of at least one of said closed loop conducting paths, said plurality of coated layers arranged so as to form, in combination with said tubular skeletal structure, an equivalent RLC circuit, said equivalent RLC circuit having a resonant frequency in the range from about 10 to about 200 megahertz and a band width in the range from about 1 to about 20 megahertz.

40. The stent as recited in claim 39, wherein said resonant frequency is in the range from about 30 to about 100 megahertz, and band width is in the range from about 3 to about 10 megahertz.

41. The stent as recited in claim 40, wherein said resonant frequency is in the range from about 40 to about 70 megahertz, and said band width is in the range from about 4 to about 7 megahertz.

42. The stent as recited in claim 39, wherein said plurality of coated layers comprises a two-layer structure comprising a conducting layer over an insulating layer, said two-layer structure disposed around said at least one of said closed loop conducting paths, in a spiral pattern, with said insulating layer adjacent to said tubular skeletal structure, thereby forming a plurality of overlapping segments of said two-layer structure.

43. The stent recited in claim 42, wherein said insulating layer has a thickness of from about 1.0 nanometer to about 1.0 millimeter, and a dielectric constant of from about 1.1 to about 2000; and said conducting layer has a conductivity greater than 1.0×10⁶ siemans/meter, and a thickness of from about 1.0 nanometer to about 1.0 millimeter.

44. The stent recited in claim 43, wherein said conducting layer is comprised of a material selected from the group consisting of aluminum, copper, gold, and silver.

45. The stent recited in claim 39, wherein said plurality of layered coatings comprises a first insulating layer adjacent to said at least one of said closed loop conducting paths, a first conducting layer over said first insulating layer, a second insulating layer over said first conducting layer, and a second conducting layer over said second insulating layer.

46. The stent recited in claim 45, wherein said first insulating layer is disposed continuously around the circumference of said at least one of said closed loop conducting paths, said first conducting layer is disposed around about 90% of the circumference of said at least one of said closed loop conducting paths, said second insulating layer is disposed continuously around the circumference of said at least one of said closed loop conducting paths, and said second conducting layer is disposed around the circumference of said at least one of said closed loop conducting paths in the range of about 30% to about 90%.

47. The stent recited in claim 46, wherein said first insulating layer has a resistivity greater than 10⁵ Ohm-centimeters, and a thickness of from about 1.0 nanometer to about 1.0 millimeter.

48. The stent recited in claim 47, wherein said second insulating layer has a thickness of from about 1.0 nanometer to about 1.0 millimeter, and a dielectric constant of from about 1.1 to about 2000.

49. The stent recited in claim 48, wherein said first conducting layer and said second conducting layer each have a conductivity greater than 1.0×10⁶ siemans/meter, and a thickness of from about 1.0 nanometer to about 1.0 millimeter.

50. The stent recited in claim 49, wherein said first conducting layer and said second conducting layer each are comprised of a material selected from the group consisting of aluminum, copper, gold, and silver.
